(19) United States
(12) Patent Application Publication
(10) Pub. No.: US 2014/0163277 A1
(43) Pub. Date: Jun. 12, 2014

(54) PROCESS FOR THE GENERATION OF 2,5-DIMETHYLHEXENE FROM ISOBUTENE

(71) Applicant: UOP LLC, Des Plaines, IL (US)

(72) Inventors: Christopher P. Nicholas, Evanston, IL (US); Stuart E. Smith, Lake Zurich, IL (US); Alakananda Bhattacharyya, Glen Ellyn, IL (US)

(73) Assignee: UOP LLC, Des Plaines, IL (US)

(21) Appl. No.: 14/102,823

(22) Filed: Dec. 11, 2013

Related U.S. Application Data
(60) Provisional application No. 61/736,098, filed on Dec. 12, 2012.

(51) Int. Cl. C07C 2/86; C07C 5/41

(52) U.S. Cl. CPC C07C 2/864; C07C 5/417; C07C 5/412
USPC 585/321; 585/639; 585/640

(57) ABSTRACT

A method of making one or more 2,5-dimethylhexenes is described. The method includes reacting isobutene with isobutanol in the presence of a platinum group metal catalyst to form one or more 2,5-dimethylhexenes. A method of making p-xylene using one or more 2,5-dimethylhexenes is also described. The p-xylene can be made from totally renewable sources, if desired.

### CROSS-REFERENCE TO RELATED APPLICATION

This application claims priority from Provisional Application No. 61/736,098 filed Dec. 12, 2012, the contents of which are hereby incorporated by reference.

### BACKGROUND OF THE INVENTION

It is known that the feed to a low-pressure reformer affects the product selectivity. For example, microreactor testing data shows that n-octane gives a mixture of ethylbenzene and o-xylene; 3-methylheptane gives a selectivity of around 50% p-xylene; and 2,5-dimethylhexane produces p-xylene with extremely high yield (e.g., approaching 95 wt % selectivity at 88% conversion).

It is also known that the aromatization of paraffins proceeds through consecutive dehydrogenations. Therefore, producing a C8 olefin with the proper 2,5-dimethyl branching structure would be desirable in a process to produce p-xylene.

Typically, a reformer will use a mixture of paraffinic feeds to achieve high yields of aromatics and hydrogen. Because of the high demand for p-xylene as a precursor for polyethylene terephthalate (PET), the yield of p-xylene from an aromatics complex drives the economics of the process. Therefore, if a feed containing substantially 2,5-dimethylhexane (or hexene) can be generated at a reasonable cost, the yield of p-xylene would be increased dramatically, and the process would be economically feasible.

### SUMMARY OF THE INVENTION

One aspect of the invention involves a method of making 2,5-dimethylhex-2-ene. In one embodiment, the method includes reacting isobutene with isobutanol in the presence of a platinum group metal catalyst to form 2,5-dimethylhex-2-ene.

Another aspect of the invention involves a method of making p-xylene. In one embodiment, the method includes reacting isobutene with isobutanol in the presence of a platinum group metal catalyst to form 2,5-dimethylhex-2-ene; and reforming the 2,5-dimethylhex-2-ene in a reforming zone under reforming conditions to form p-xylene.

### BRIEF DESCRIPTION OF THE DRAWINGS

FIG. 1 is a diagram of a prior art pathway from isobutene to p-xylene,

FIG. 2 is a diagram of an acid catalyzed mechanism for isobutene dimerization.

FIG. 3 is a diagram of a base catalyzed mechanism for isobutene dimerization.

FIG. 4 is a diagram of the synthesis of 2,5-dimethylhexene from isobutene and isobutanol according to one embodiment of the present invention.

FIG. 5 is a diagram of a process of forming p-xylene from isobutene and isobutanol according to one embodiment of the present invention.

### DETAILED DESCRIPTION OF THE INVENTION

P-xylene can be generated in high selectivity by reforming 2,5-dimethylhexane (>80 wt % selectivity, U.S. Pat. No. 6,358,400 B1 and U.S. Pat. No. 6,177,601 B1). Since the process is believed to proceed via sequential dehydrogenation, any dimethylhexene possessing branching in the 2 and 5 positions should also selectively reform to produce p-xylene. 2,5-dimethylhexene, as defined herein, is taken to mean all octene isomers possessing branching in the 2 and 5 positions and includes 2,5-dimethylhex-1-ene, 2,5-dimethylhex-2-ene, cis-2,5-dimethylhex-3-ene and trans-2,5-dimethylhex-3-ene.

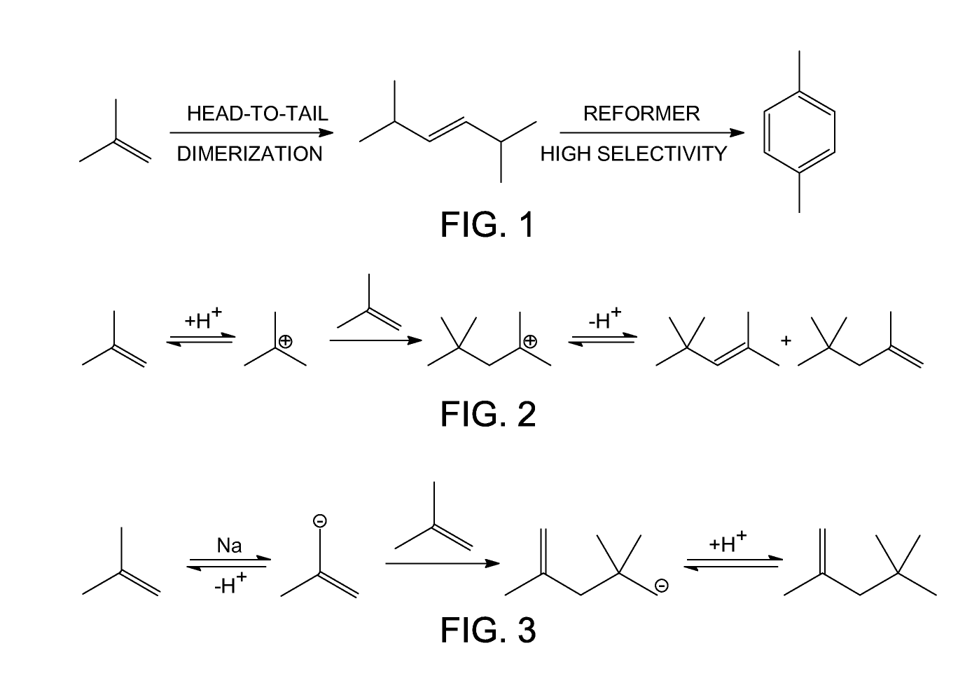

P-xylene can be generated through the head-to-tail dimerization of isobutene to 2,5-dimethylhexene followed by a reforming step, as shown in FIG. 1. However, the selectivity of isobutene head-to-tail dimerization using existing catalysts, which ranges as high as about 20 to 30%, is too low to make the process economically viable.

Previous studies have shown low selectivity to the desired 2,5-dimethylhexene component. Dimerization of isobutene often yields multiple products. For example, dimerization of butenes over acid catalysts, such as solid phosphoric acid (SPA), yields a number of products, including significant fractions of trimethylpentene. The primary dimethyl C₈ made over an acid catalyst is 3,4-dimethylhexene. Acid catalysts are expected to yield high quantities of trimethylpentenes due to carbocation stability as shown in FIG. 2. Typically, significant isomerization occurs, giving a yield of many products. Base-catalyzed dimerizations should also yield high selectivity to trimethylpentenes due to carbanion stability as shown in FIG. 3.

The coupling of an alkene with an alcohol to form a coupled alkene with the same number of carbon atoms as the combined feed, and water has been demonstrated. Lee, D. H.; Kwon, K. H.; and Yi, C. S., “Selective Catalytic C—H Alkylation of Alkenes with Alcohols”, SCIENCE 2011, 333, 1613-6. For example, propylene combined with ethanol formed primarily 2-pentene and water with a small amount of 2-methylbutene. Yi studied the reaction primarily utilizing cyclic olefins to determine which substrates can be used in the process. No reactions involving iso-olefins such as isobutene were described.

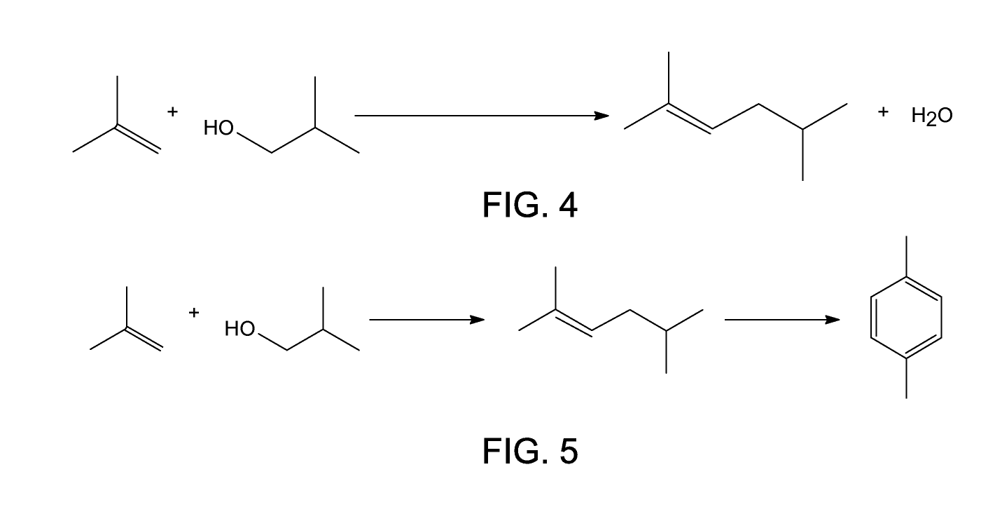

FIG. 4 illustrates the reaction of isobutene with isobutanol to form 2,5-dimethylhex-2-ene as the primary product. Suitable reaction conditions include a temperature in the range of about 25° to about 500° C., and a pressure in the range of about 101 kPa (1 atm) to about 10.1 mPa (10 atm).

The catalyst for the reaction is a platinum group metal catalyst. Suitable platinum group metals include platinum, ruthenium, rhodium, palladium, osmium, and iridium. A ruthenium catalyst can be used, for example. The ruthenium catalyst can be a cationic ruthenium center catalyst. Suitable ruthenium catalysts include, but are not limited to, Ru/C catalysts, Ru/Al₂O₃ catalysts, or combinations thereof. Additionally, the ruthenium catalyst can be derived from the catalyst precursors [(C₆H₆)(PCy₃)(CO)RuH]⁺BE₄⁻(C₆H₆=benzene, PCy₃=tricyclohexylphosphine, CO=carbon monoxide and H=hydride), (PCy₃)₂(CO)RuH(μ-OH)(μ-H)(PCy₃)(CO)RuH (where μ indicates the respective ligand is bridging two metals, μ₃ indicates the respective ligand is bridging three metals and μ₄ indicates the respective ligand is bridging four metals), {[(PCy₃)(CO)RuH]₄(μ₄-O)(μ₃-OH)(μ₂-OH)} and (p-cymene)(PCy₃)RuCl₂. The catalyst precursors (PCy₃)₂(CO)RuH(μ-OH)(μ-H)(PCy₃)(CO)RuH, activated with HBF₄.Et₂O (Et₂O=diethyl ether), and (p-cymene)(PCy₃)RuCl₂, activated with AgBF₄, used in the following embodiments have been shown to be effective in producing 2,5-dimethylhex-2-ene. Despite their vastly different structures, these compounds are capable of producing the same compound. A common feature of these ruthenium compounds is the presence of PCy₃, H and CO. In one embodiment, the catalyst is [(C₆H₆)(PCy₃)(CO)RuH]⁺BE₄⁻(PCy₃=tricyclohexylphosphine). In another embodiment, the catalyst is the binuclear complex (PCy₃)₂(CO)RuH(μ-OH)(μ-H)(PCy₃)(CO)RuH and HBF₄.Et₂O (Et₂O=diethyl ether). In yet another embodiment, the catalyst is generated from (p-cymene)(PCy₃)RuCl₂ and AgBF₄. It should be possible to generate an active catalyst from {[(PCy₃)(CO)RuH](μ₄-O)(μ₃-OH)(μ₂-OH)} and HBF₄.Et₂O.

In some embodiments when [(C₆H₆)(PCy₃)(CO)RuH]⁺BE₄⁻ is used as the catalyst, suitable reaction conditions can include temperatures ranging from about 75° to about 150° C. for about 2 to about 8 hours. In another embodiment, when the catalyst is the binuclear complex (PCy₃)₂(CO)RuH(μ-OH)(μ-H)(PCy₃)(CO)RuH and HBF₄.Et₂O, suitable reaction conditions can include temperatures ranging from about 75° to about 150° C. for about 1 to about 48 hours. In yet another embodiment, when the catalyst is generated from (p-cymene)(PCy₃)RuCl₂ and AgBF₄, suitable temperatures can range from about 75° to about 150° C. for about 1 to about 48 hours.

The ruthenium catalyst precursors (PCy₃)₂Ru(H)(Cl)(CO), (PCy₃)₂(CO)RuH(μ-OH)(μ-H)(PCy₃)(CO)RuH and {[(PCy₃)(CO)RuH]₄(μ₄-O)(μ₃-OH)μ₂-OH)} can be synthesized at ambient pressures in contrast to previously reported synthetic procedures. The reported synthetic procedures took place in sealed glass vessels above the solvents boiling point, which result in positive pressure within a sealed glass vessel and introduce safety concerns. The solvents in these reported reactions also react with the ruthenium reactant to form the desired product, thus simply replacing one solvent for a higher boiling solvent does not necessarily mean that the desired compound will be formed. In order to synthesize these ruthenium compounds at ambient pressure, the new solvent must possess both a boiling point equal to or greater than the reaction temperature, but also still be reactive with the ruthenium reactant. The synthesis of (PCy₃)₂Ru(H)(Cl)(CO) occurs by reacting [(COD)RuCl]ₙ (COD=1,5-cyclooctadiene) and PCy₃ in n-propanol at 95° C. The synthesis of (PCy₃)₂(CO)RuH(μ-OH)(μ-H)(PCy₃)(CO)RuH occurs by reacting (PCy₃)₂Ru(H)(Cl)(CO) and KOH in 2-propanol at 85° C. The synthesis of {[(PCy₃)(CO)RuH]₄(μ₄-O)(μ₃-OH)(μ₂-OH)} occurs by reacting (PCy₃)₂(CO)RuH(μ-OH)(μ-H)(PCy₃)(CO)RuH in 2-hexanone at 95° C. The synthesis of [(C₆H₆)(PCy₃)(CO)RuH]⁺BE₄⁻ occurs by reacting {[(PCy₃)(CO)RuH]₄(μ₄-O)(μ₃-OH)(μ₂-OH)} with HBF₄.Et₂O in benzene at room temperature. In other embodiments when heterogeneous catalysts are used, higher temperatures may be potentially useful.

The ruthenium catalyst can be mononuclear, binuclear, trinuclear, tetranuclear or contain n ruthenium atoms, where n=1-100,000. Additionally, the ruthenium complex can be nanocluster, cluster or bulk ruthenium. Ruthenium catalyst precursors can be [(C₆H₆)(PCy₃)(CO)RuH]⁺BE₄⁻, (PCy₃)₂(CO)RuH(μ-OH)(μ-H)(PCy₃)(CO)RuH, {[(PCy₃)(CO)RuH]₄(μ₄-O)(μ₃-OH)(μ₂-OH)} and (p-cymene)(PCy₃)RuCl₂ (PCy₃=tricyclohexylphosphine). It is known that mononuclear precursors can decompose in situ to generate nanoclusters or bulk metallic species (see Widegren, J. A.; Bennet, M. A.; Finke, R. G. J. AM. CHEM. SOC. 2003, 125, 10301; Finney, E. E. J. COLLOID INTERFACE SCI. 2008, 317, 351 and Lin, Y.; Finke, R. G. J. AM. CHEM. SOC. 1994, 116, 8335). The ruthenium catalyst can be ligated by several ligands; typical ligands are PCy₃, CO, H and arenes. The ruthenium catalyst can be charged with a typical counter ion being BF₄⁻.

The isobutene source could be any of the traditional petroleum based C₄ sources, or renewable sources such as dehydrated isobutanol. Isobutene can be found in C₄ streams such as that obtained from fluidized catalytic cracking. Isobutene is in relatively high supply currently due to the phase-out of methyl t-butyl ether (MTBE) production. Alternatively, underutilized isobutane can be converted to isobutene using catalytic dehydrogenation technology, as described, for example, in U.S. Pat. No. 7,439,409, which is incorporated herein. Additionally, bio-derived isobutanol is coming onto the market, yielding another potential source of isobutene via dehydration.

The isobutanol may come from traditional carbon sources such as syngas by the conversion of methanol, ethanol or propanol using the Guerbet reaction in the presence of catalysts such as hydrotalcites (Carlini et al, “Guerbet condensation of methanol with n-propanol to isobutyl alcohol over heterogeneous bifunctional catalysts based on Mg—Al mixed oxides partially substituted by different metal compounds,” J. MOL. CATAL. A 2005, 232, 13-20) or copper chromite and sodium methoxide (Carlini et al., “Selective synthesis of isobutanol by means of the Guerbet reaction Part 1. Methanol/n-propanol condensation by using copper based catalytic systems,” J. MOL. CATAL. A 2002, 184, 273-280). The isobutanol could come from renewable sources such as bio-derived sources or hydrated isobutene.

Renewable sources include, but are not limited to, fermentation of renewable carbon sources of glucose, sucrose, fructose, monosaccharides, oligosaccharides, polysaccharides, and mixtures thereof, conversion of renewable carbon sources of methanol, ethanol, propanol and combinations thereof to isobutanol and mixtures of these sources. Renewable carbon sources are those carbon sources which can be grown, harvested and replanted in a short time period, unlike petroleum which takes millions of years to form. Examples thereof include, but are not limited to switch grass, corn, sugar cane and jatropha.

The reaction may take place in the presence of a solvent. Suitable solvents include, but are not limited to isobutanol and chlorinated solvents, or combinations thereof. Chlorinated solvents are organic compounds that are liquid at the reaction temperature and which possess only C, Cl and/or H atoms present.

Feeding isobutene and isobutanol over the ruthenium catalyst can yield high selectivity to 2,5-dimethylhex-2-ene. The reaction can have a selectivity for 2,5-dimethylhex-2-ene of greater than about 1%, or greater than about 5%, or greater than about 10%, or greater than about 15%, or greater than about 20%, or greater than about 25%, or greater than about 50%, or greater than about 75%, or greater than about 80%, or greater than about 85%, or greater than about 90%, or greater than about 95%.

A process for making p-xylene from isobutene and isobutanol is illustrated in FIG. 5. The isobutene and isobutanol are reacted as described above to form 2,5-dimethylhex-2-ene, which is then subjected to a catalytic reforming process to form p-xylene.

Catalytic reforming processes use a catalyst comprising a Group VIII noble metal on a support to convert the 2,5-dimethylhex-2-ene to p-xylene. Suitable operating conditions include a pressure of from about 100 kPa to about 1.0 MPa (absolute), or about 100 to about 500 kPa, or a pressure of below about 300 kPa. Free hydrogen optionally is supplied to the process in an amount sufficient to correspond to a ratio of from about 0.1 to about 10 moles of hydrogen per mole of hydrocarbon feedstock. By “free hydrogen” is meant molecular H₂, not combined in hydrocarbons or other compounds. Preferably, the reaction is carried out in the absence of added halogen. The volume of catalyst corresponds to a liquid hourly space velocity of from about 0.5 to about 40 hr⁻¹. The operating temperature generally is in the range of about 260° to about 600° C. Temperature selection is influenced by product objectives, with higher temperatures effecting higher conversion of the feedstock to aromatics. Hydrocarbon types in the feedstock also influence temperature selection, as naphthenes are largely dehydrogenated over the first portion of the reforming catalyst which the feedstock contacts with a concomitant sharp decline in temperature across the first catalyst bed due to the endothermic heat of reaction. The temperature generally is slowly increased during each period of operation to compensate for inevitable catalyst deactivation.

The wt % selectivity to p-xylene from one or more 2,5-dimethylhexenes can be greater than about 50%, or greater than about 55%, or greater than about 60%, or greater than about 65%, or greater than about 70%, or greater than about 75%, or greater than about 75%, or greater than about 80%.

The reforming process may be affected in a reactor zone comprising one reactor or in multiple reactors with provisions known in the art to adjust inlet temperatures to individual reactors. The feed may contact the catalyst system in each of the respective reactors in either upflow, downflow, or radial-flow mode. Since the preferred reforming process operates at relatively low pressure, the low pressure drop in a radial-flow reactor favors the radial-flow mode. As the predominant dehydrocyclization and dehydrogenation reactions are endothermic, the reactor section generally will comprise two or more reactors with interheating between reactors to compensate for the endothermic heat of reaction and maintain dehydrocyclization conditions.

Using techniques and equipment known in the art, the aromatics-rich effluent usually is passed through a cooling zone to a separation zone. In the separation zone, typically maintained at about 0° to 65° C., a hydrogen-rich gas is separated from a liquid phase. The resultant hydrogen-rich stream can then be recycled through suitable compressing means back to the first reforming zone. The liquid phase from the separation zone is normally withdrawn and processed in a fractionating system in order to adjust the concentration of light hydrocarbons and produce an aromatics-containing reformate product.

The reactor section usually is associated with catalyst-regeneration options known to those of ordinary skill in the art, such as: (1) a semiregenerative unit containing fixed-bed reactors which maintains operating severity by increasing temperature, eventually shutting the unit down for catalyst regeneration and reactivation; (2) a swing-reactor unit, in which individual fixed-bed reactors are serially isolated by manifolding arrangements as the catalyst becomes deactivated, and the catalyst in the isolated reactor is regenerated and reactivated while the other reactors remain on-stream; (3) continuous regeneration of catalyst withdrawn from a moving-bed reactor, with reactivation and substitution of the reactivated catalyst, permitting higher operating severity by maintaining high catalyst activity through regeneration cycles of a few days; or (4) a hybrid system with semiregenerative and continuous-regeneration provisions in the same unit.

Reforming catalysts generally comprise a metal on a support. The support can include a porous material, such as an inorganic oxide or a molecular sieve, and a binder with a weight ratio from 1:99 to 99:1. The weight ratio is preferably from about 1:9 to about 9:1.

The metals preferably are one or more Group VIII noble metals, and include platinum, iridium, rhodium, and palladium. The Group VIII noble metals may exist within the final catalytic composite as a compound such as an oxide, sulfide, halide, or oxyhalide, in chemical combination with one or more of the other ingredients of the composite, or as an elemental metal. Better results may be obtained when substantially all of the metals are present in the elemental state. The Group VIII noble metal component may be present in the final catalyst composite in any amount which is catalytically effective, but relatively small amounts are preferred. Typically, the catalyst contains an amount of the metal from about 0.01% to about 2% by weight, based on the total weight of the catalyst. The catalyst can also include a promoter element from Group IIIIA or Group IVA. These metals include gallium, germanium, indium, tin, thallium and lead.

Inorganic oxides used for support include, but are not limited to, alumina, magnesia, titania, zirconia, chromia, zinc oxide, thoria, boria, ceramic, porcelain, bauxite, silica, silica-alumina, silicon carbide, clays, crystalline zeolitic aluminosilicates, and mixtures thereof. Porous materials and binders are known in the art and are not presented in detail here.

In an embodiment, the Group VIII noble metal is supported on a bound molecular sieve. Suitable molecular sieves generally have a maximum free channel diameter or “pore size” of 6 Å or larger, and preferably have a moderately large pore size of about 7 to 8 Å. Such molecular sieves include those characterized as AFI, BEA, ERI, FAU, FER, LTL or MWW structure type by the IUPAC Commission on Zeolite Nomenclature. The zeolite is typically combined with a binder in order to provide a convenient form for use in the catalyst particles of the present invention.

The Group VIII noble metal component may be incorporated in the porous carrier material in any suitable manner, such as co-precipitation, ion-exchange or impregnation.

The reforming catalyst may contain a halogen component. An optional halogen component may be fluorine, chlorine, bromine, iodine or mixtures thereof. An optional halogen component is generally present in a combined state with the inorganic-oxide support, and preferably is well distributed throughout the catalyst and may comprise from more than 0.2 to about 15 mass-% calculated on an elemental basis, of the final catalyst.

The high selectivity to 2,5-dimethylhex-2-ene provides a new, economically attractive pathway to form p-xylene. In addition, if the isobutene and isobutanol are derived from renewable sources, the p-xylene could be made from entirely renewable sources.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

### EXAMPLES

Unless otherwise noted, all reactions and manipulations were carried out under a N₂ atmosphere using standard Schlenk and high-vacuum line techniques, or in an inert atmosphere glove box (N₂) at ambient temperature.

¹H, ¹³C{¹H}, ³¹P{1H} NMR spectra were recorded at 500, 125, and 202 MHz respectively on a Bruker 500 MHz Avance III spectrometer. All NMR chemical shifts are reported as 6 in parts per million (ppm). All NMR spectra were acquired at room temperature. ¹H NMR spectra were referenced to residual protiated solvent, and chemical shifts are reported in parts per million downfield from tetramethylsilane. ³¹P {¹H} NMR spectra are reported relative to 85% H₃PO₄ as the external standard. ¹³C {¹H} NMR spectra were referenced to solvent.

Unless otherwise noted, reagents were purchased from commercial suppliers and used without further purification. Solvents were degassed by sparging with nitrogen prior to use. Dichloromethane was purified by washing with 5% sodium bicarbonate solution, followed by washing with an equal volume of distilled water. After separation, the dichloromethane was dried over anhydrous MgSO₄, filtered and dried over activated 3A molecular sieves (10% m/v). Dichloromethane was then degassed by sparing with nitrogen for 40 minutes and then stored under nitrogen.

All GC data were acquired on an Agilent 7890A using a 50 m×200 μm×0.5 μm PONA column. The hydrogen flow rate was kept constant at 1.1 mL/min. The initial oven temperature was 50° C. without a hold time and was then ramped to 110° C. at 10° C./minute and then immediately ramped to 300° C. at 20° C./minute and held at 300° C. for 5 minutes. Typical retention times (minutes) are: 2.71 (isobutylene), 4.00 (isobutanol), 5.25 (2,4,4-trimethylpent-1-ene), 5.44 (2,4,4-trimethylpent-2-ene), 5.97 (2,5-dimethylhex-2-ene) and 9.22 (n-decane) and were determined by injecting known compounds onto the GC. Products were quantified using n-decane as the internal standard and using the effective carbon numbers as reported in Scanlon, J. T.; Willis, D. E., J. CHROMATOGR. SCI. 1985, 23, 333. Oxygen containing compounds were identified by GC/MS. For the oxygen containing compounds that were identified by GC/MS, quantification was based on the GC chromatogram using the FID detector in conjunction with the effective carbon numbers. Typical retention times (minutes) for the assigned oxygenated compounds are: 2-methyl-1,1-bis(2-methylpropoxy)propane (10.41), 1-tert-butoxy-2-methylpropane (5.81) and 2-methylpropyl 2-methylpropanoate (7.92). Selectivity is determined from the % isobutanol conversion and by determining the number of isobutanol units that make up a given product. For example, the products 2,5-dimethylhex-2-ene would be composed of 1 isobutanol, the product 2-methyl-1,1-bis(2-methylpropoxy)propane would be composed of 3, the product 1-tert-butoxy-2-methylpropane is composed of 1 unit, the product and 2-methylpropyl 2-methylpropanoate is composed of 2 units. GC/MS data were acquired on an Agilent technologies 5975B GC/MS using a 50 m×200 μm×0.5 μm PONA column. The hydrogen flow rate was kept constant at 0.3 mL/min. The initial oven temperature was 35° C. with an 8 minute hold time, which was then ramped to 240° C. at 5° C./minute and then held at this temperature for 15 minutes.

### Example 1

### Synthesis of (PCy₃)₂(CO)RuH(μ-OH)(μ-H)(PCy₃)(CO)RuH

A 100 mL Schlenk tube equipped with a stir bar was charged with (PCy₃)₂Ru(H)(Cl)(CO) (2.04 g, 2.8 mmoles) and KOH (1.14 g, 20.3 mmoles) in a nitrogen glovebox. The flask was sealed with a rubber septum, removed from the glovebox, attached to a Schlenk line and put under nitrogen. Isopropyl alcohol was sparged with nitrogen for 1 hour 15 minutes and then added to the Schlenk flask (26 mL) via syringe. A reflux condenser was attached to the Schlenk flask and the mixture was stirred over night at room temperature under nitrogen in an oil bath. The following day, the oil bath was heated to 85° C., which took about 1 hour to reach temperature, and then stirred at this temperature for 8 hours. After this time, the reaction mixture was cooled to room temperature and the volatile components were removed under vacuum on the Schlenk line. The resulting yellow solid was stored under nitrogen. The following day, the yellow solid was washed 3× with isopropyl alcohol, which had been pre-sparged with nitrogen for 40 minutes. The first and third washes used about 26 mL and the second about 40 mL. For the first and second wash, the solid was stirred in the isopropyl alcohol for about 5 minutes before cannula transferring the isopropyl alcohol washes away. The third wash used about 26 mL and the solution was stirred for 1.5 hours before cannula transferring the wash away. The remaining solid was dried on the Schlenk line under vacuum, yielding 1.62 g of a yellow solid. This solid contains residual isopropyl alcohol in a 0.56:1.00 molar ratio of isopropyl alcohol:ruthenium complex, as determined by ¹H NMR spectroscopy. NMR data for (PCy₃)₂(CO)RuH(μ-OH)(μ-H)(PCy₃)(CO)RuH: ¹H NMR matches that reported in Yi, C. S.; Zeczycki, T. N.; Guzei, I. A., ORGANOMETALLICS 2006, 25, 1047. The ³¹P {¹H} NMR spectrum did not match the reported values, the ³¹P {¹H} NMR spectrum we observed are reported as follows: (500 MHz, CD₂Cl₂): δ 65.62 (d, J_{P-P}=282 Hz), 54.17 (d, J_{P-P}=282 Hz) and 53.4 (t, J_{P-P}=3 Hz).

### Example 2

### Synthesis of (PCy₃)₂Ru(H)(Cl)(CO)

An oven-dried 200 mL Schlenk flask equipped with a stir bar was charged with [(COD)RuCl]ₙ (2.049 g, 7.3 mmoles) and PCy₃ (4.095 g, 14.6 mmoles) in a nitrogen glovebox. The flask was sealed with a rubber septum, removed from the glovebox, attached to a Schlenk line and n-propanol (70 mL) was added via syringe. A reflux condenser was attached to the flask and the reaction mixture heated to 95° C. using an oil bath. The reaction was stirred and this temperature and maintained under a nitrogen atmosphere for 46 hours. During this time, an orange precipitate formed and the mother liquor was deep brown, nearly black in color. The reaction mixture was then cooled to room temperature and the mother liquor cannula transferred away from the precipitate. The precipitate was then washed with 3×20 mL n-propanol and the washings were separated by cannula filtration. The remaining volatile compounds were removed under vacuum yielding 2.30 g of product. NMR data for (PCy₃)₂Ru(H)(Cl)(CO): ¹H and ³¹P {¹H} NMR matches that reported in Yi, C. S.; Lee, D. W.; Chen, Y., ORGANOMETALLICS 1999, 18, 2043. Impurities are present in the ³¹P {¹H} NMR spectrum located at 50.5 (5%), 49.5 (<1%) and 35.6 (3%). The percentages are qualitative and were determined from the ³¹P {¹H} NMR spectrum, Ti values were not measured.

### Example 3

### Synthesis of {[(PCy₃)(CO)RuH]₄(μ₄-O)(μ₃-OH)(μ₂-OH)}

The ruthenium compound (PCy₃)₂(CO)RuH(μ-OH)(μ-H)(PCy₃)(CO)RuH (0.51 g, 0.46 mmoles) and 2-hexanone (5 mL, pre-sparged with nitrogen) were added to a vial in the glovebox. The ruthenium compound was slurried in the 2-hexanone and the slurry transferred to an oven-dried 50 mL Schlenk flask equipped with a stir bar. The flask was stoppered with a rubber septum, removed from the glovebox and attached to a Schlenk line. A reflux condenser was attached to the Schlenk flask and the reaction mixture was heated to 95° C. under nitrogen. Once the reaction temperature was reached (about 45 minutes), the reaction was stirred for 2.8 hours; during this time, the solid changed from yellow to red. The reaction mixture was then cooled to room temperature and the volatile components were removed under vacuum, yielding a red-brown solid. The solid was washed with 1×10 mL acetone (sparged with nitrogen for about 40 minutes) and washed with 3×5 mL 2-propanol (pre-sparged with nitrogen for 40 minutes). The solid was then dissolved in dichloromethane and a small amount of benzene was added to the solution. The solution was then concentrated under vacuum and once sufficiently concentrated, 2-propanol was added to the solution and a solid then precipitated out of solution. The mother liquor was cannula transferred away from the solid and the solid was dried on the Schlenk line, which yielded 0.16 g of a reddish-brown solid. NMR data for {[(PCy₃)(CO)RuH]₄(μ₄-O)(μ₃-OH)μ₂-OH)}: ¹H NMR matches that reported in Yi, C. S.; Zeczycki, T. N.; Guzei, I. A. ORGANOMETALLICS 2006, 25, 1047. Residual 2-propanol is present in a 1.0:1.0 ratio relative to a single Ru—H resonance in the product. Impurities are present in the ¹H NMR spectrum and the concentrations are listed as ratios relative to a single hydride resonance in the ruthenium product. These impurities are located at −4.23 (s, 0.06:1.00), −4.43 (s, 0.06:1.00), −11.50 (pseudo d, J_{P-H}=20 Hz, 0.06:1.00), −12.92 (pseudo d, J_{P-H}=35 Hz, 0.07:1.00), −16.51 (pseudo d, J_{P-H}=16 Hz, 0.06:1.00), −17.85 (t, J_{P-H}=19 Hz, 0.12:1.00) and −17.86 (t, J_{P-H}=19 Hz, 0.08:1.00). The ³¹P {¹H} NMR spectrum did not match the reported values, the ³¹P {¹H} NMR spectrum we observed are reported as follows: (500 MHz, CD₂Cl₂): δ 81.83 (s), 78.50 (s), 71.50 (s) and 68.41 (s). Impurities are present in the ³¹P {¹H} NMR spectrum, these impurities are reported as ratios relative to a single ³¹P resonance of the product. These impurities are located at 79.01 (s, 0.03:1.0), 76.82 (s, 0.06:1.0), 71.24 (s, 0.04:1.0), 55.67 (s, 0.10:1.00), 49.44 (s, 0.18:1.00), 46.22 (s, 0.03:1.00), 45.84 (s, 0.20:1.00), 45.58 (s, 0.06:1.00), 45.25 (s, 0.17:1.00) and 11.14 (s, 0.05:1.00).

### Example 4

### Synthesis of {[(PCy₃)(CO)Ruh]₄(μ₄-O)μ₃-OH)(μ₂-OH)}

The ruthenium compound (PCy₃)₂(CO)Ruh(μ-OH)(μ-H)(PCy₃)(CO)RuH (0.53 g, 0.53 mmoles) was massed into a glass insert equipped with a stir bar in the glovebox. To this insert was added acetone (5 mL, pre-sparged with nitrogen). The glass insert was transferred to a 75 mL Hastelloy C autoclave, which was then sealed and removed from the glovebox. The reaction mixture was then heated to 95° C. with an oil bath and stirred at this temperature for 3 hours. Afterwards, the autoclave was cooled and brought back into the glovebox. The solution was then filtered through a plug of celite and the solid washed with 2×5 mL 2-propanol. The remaining solid was dissolved in dichloromethane (used as is, after degassing) and collected. The solution was concentrated under vacuum and crystallized at −78° C. The mother liquor was removed with a syringe and the precipitate was dried under vacuum yielding 0.04 g. NMR data for {[(PCy₃)(CO)Ruh]₄(μ-O)(μ₃-OH)(μ₂-OH)}: ¹H NMR matches that reported in Yi, C. S.; Zeczycki, T. N.; Guzei, I. A. ORGANOMETALLICS 2006, 25, 1047. Residual 2-propanol is present in a 1.6:1.0 ratio relative to a single Ru—H resonance in the product. Impurities are present in the ¹H NMR spectrum and the concentrations are listed as ratios relative to a single hydride resonance in the ruthenium product. These impurities are located at −0.47 (s, 0.04:1.0), −0.64 (s, 0.01:1.00), −0.68 (s, 0.03:1.00), −0.89 (s, 0.18:1.00), −1.14 (s, 0.03:1.00), −1.40 (s, 0.04:1.00), −1.58 (s, 0.04:1.00), −3.88 (s, 0.04:1.00), −4.24 (s, 0.17:1.00), −4.30 (s, 0.01:1.00), −4.44 (s, 0.17:1.00), −11.52 (pseudo d, J_{P-H}=19 Hz, 0.18:1.00), −12.93 (pseudo d, J_{P-H}=34 Hz, 0.17:1.00), −13.15 (pseudo d, J_{P-H}=34 Hz, 0.02:1.00), −16.52 (pseudo d, J_{P-H}=16 Hz, 0.18:1.00), −16.93 (pseudo d, J_{P-H}=18 Hz, 0.07:1.00) and −17.87 (t, J_{P-H}=19 Hz, 1.28:1.00). The complex (PCy₃)₂Ru(H)(Cl)(CO) is also observed in a 1.6:1.0 molar ratio. The ³¹P {¹H} NMR spectrum did not match the reported values, the ³¹P {¹H} NMR spectrum we observed are reported as follows: (500 MHz, CD₂Cl₂): δ 81.83 (s), 78.50 (s), 71.50 (s) and 68.41 (s). Impurities are present in the ³¹P {¹H} NMR spectrum, these impurities are reported as ratios relative to a single ³¹P resonance of the product. These impurities are located at 81.15 (s, 0.08:1.00), 79.02 (s, 0.18:1.00), 76.84 (s, 0.18:1.00), 76.46 (s, 0.04:1.00), 71.24 (s, 0.20:1.00), 55.68 (s, 0.21:1.00), 49.74 (s, 0.08:1.00), 47.17 (s, 0.03:1.00), 45.27 (s, 2.74:1.00), 34.14 (s, 0.03:1.00). The complex (PCy₃)₂Ru(H)(Cl)(CO) is also observed in the ³¹P {¹H} NMR spectrum in a 1.8:1.0 molar ratio. The [(C₆H₆)(PCy₃)Ru(H)(CO)][BF₄] synthesized from this complex using the procedure described in Example 5 yielded a product containing the impurity [H—PCy₃][BF₄] in a 0.6:1.0 molar ratio of [H—PCy₃][BF₄]:[(C₆H₆)(PCy₃)Ru(H)(CO)][BF₄].

### Example 5

### Synthesis of [(C₆H₆)(PCy₃)Ru(H)(CO)][BF₄]

The ruthenium compound {[(PCy₃)(CO)RuH]₄(μ₄-O)(μ₃-OH)(μ₂-OH)} (0.160 g, 0.09 mmoles) was added to a 50 mL Schlenk flask equipped with a stir bar and 10 mL benzene (pre-sparged with nitrogen for 40 min). The flask was removed from the glovebox, attached to the Schlenk line and HBF₄.Et₂O (50 μL, 0.36 mmoles) was added under nitrogen. The solution became yellow and trace amounts of a precipitate formed. The reaction was stirred at room temperature for 1.5 hours and then the volatile compounds were removed under vacuum. The solid was yellow with a slight greenish-brown color to it. The crude solid was dissolved in dichloromethane, filtered and dried under vacuum. The resulting solid was taken up again in dichloromethane about 10 mL) and hexanes about 30 mL, pre-sparged with nitrogen and dried over activated 3A molecular sieves) and a red oil crashed out of solution. The mother liquor was separated from the red oil and the red oil was dried on the vacuum line, yielding the product. ¹H and ³¹P {¹H} NMR matches that reported in Yi, C. S.; Lee, D. W. ORGANOMETALLICS 2010, 29, 1883. The product contains [H—PCy₃][BF₄] as an impurity in a 0.13:1.00 ratio to the Ru complex based on the ¹H NMR. This ratio is also observed in the ³¹P {¹H} NMR spectrum.

### Example 6

### Synthesis of 2,5-dimethylhex-2-ene from isobutanol, isobutylene, (PCy₃)₂(CO)RuH(μ-OH)(μ-H)(PCy₃)(CO)RuH and HBF₄.Et₂O (1:2 molar ratio)

A stock solution consisting of isobutanol (46.8 wt %), n-decane (0.7 wt %) and chlorobenzene (52.5 wt %) was prepared. A portion of the stock solution (6.7071 g stock solution, 42.3 mmoles isobutanol) was transferred to a Schlenk flask followed by 67 μL of HBF₄.Et₂O (0.5 mmoles). The isobutanol/chlorobenzene/n-decane/HBFEt₂O mixture was then degassed by 3× freeze/pump/thaw cycles. In the glovebox, the ruthenium compound (PCy₃)₂(CO)RuH(μ-OH)(μ-H)(PCy₃)(CO)RuH (0.2042 g, 0.24 mmoles) was massed into an oven-dried 75 mL Hastelloy C autoclave equipped with a stir bar, followed by the isobutanol solution. The autoclave was then sealed, removed from the glovebox and isobutylene (2.3 g, 41 mmoles) was charged into the autoclave. The autoclave was then place in a 100° C. oil bath and the reaction mixture stirred at this temperature for 24 hours. Afterwards, the autoclave was cooled to room temperature, vented and opened. An aliquot was removed from the reactor, filtered through a plug of SiO₂, the SiO₂ plug was then flushed with an equal volume of 4% methanol in dichloromethane and the dichloromethane/methanol flush combined with the reaction filtrate was then analyzed by GC using the method listed above. GC analysis revealed that the product 2,5-dimethylhex-2-ene had formed, but was present in small amounts. Confirmation of its existence was confirmed by GC/MS and by spiking the product mixture with known 2,5-dimethylhex-2-ene. Quantification revealed that the product formed in <1% selectivity. The primary products that formed were identified by GC/MS. The primary products were 2-methyl-1,1-bis(2-methylpropoxy)propane, 1-tert-butoxy-2-methylpropane and 2-methylpropyl 2-methylpropanoate and were formed in 46%, 39% and 6% selectivity at 43% isobutanol conversion.

### Example 7

### Synthesis of 2,5-dimethylhex-2-ene from isobutanol, isobutylene, (PCy₃)₂(CO)RuH(μ-OH)(μ-H)(PCy₃)(CO)RuH and HBF₄.Et₂O (1.0:1.5 molar ratio)

A stock solution consisting of isobutanol (46.82 wt %), n-decane (0.72 wt %) and chlorobenzene (52.46 wt %) was prepared. A portion of the stock solution (7.1423 g stock solution, 45.1 mmoles isobutanol) was transferred to a Schlenk flask and then 60 μL of HBF₄.Et₂O (0.44 mmoles). The isobutanol/chlorobenzene/n-decane/HBFEt₂O mixture was then degassed by 3× freeze/pump/thaw cycles. In the glovebox, the ruthenium compound (PCy₃)₂(CO)RuH(μ-OH)(μ-H)(PCy₃)(CO)RuH (0.2498 g, 0.30 mmoles) was massed into an oven-dried 75 mL Hastelloy C autoclave equipped with a stir bar, followed by the isobutanol solution. The autoclave was then sealed, removed from the glovebox and isobutylene (1.9 g, 34 mmoles) was charged into the autoclave. The autoclave was then place in a 100° C. oil bath and the reaction mixture stirred at this temperature for 24 hours. Afterwards, the autoclave was cooled to room temperature, vented and opened. An aliquot was removed from the reactor, filtered through a plug of Celite diatomaceous earth and analyzed by GC using the method listed above. GC analysis revealed that the product 2,5-dimethylhex-2-ene had formed, but was present in small amounts. Quantification revealed that the product formed in <1% selectivity. The primary products that formed were identified by GCMS. The primary products were 2-methyl-1,1-bis(2-methylpropoxy)propane, 1-tert-butoxy-2-methylpropane and 2-methylpropyl 2-methylpropanoate and were formed in 52%, 10% and 10% selectivity at 26% isobutanol conversion.

### Example 8

### Synthesis of 2,5-dimethylhex-2-ene from isobutanol, isobutylene, (p-cymene)(PCy₃)RuCl₂ and AgBF₄

A stock solution consisting of isobutanol (18.06 wt %), n-decane (1.04 wt %) and chlorobenzene (80.90 wt %) was prepared. The stock solution was sparged with nitrogen 42 minutes. (p-cymene)(PCy₃)RuCl₂ (0.050 g, 0.085 mmoles) was charged into a vial in the glovebox and AgBF₄ (0.034 g, 0.175 mmoles) was added to a separate vial. The ruthenium compound was dissolved in chlorobenzene (2.265 g), producing a red solution. The ruthenium solution was then transferred to the vial containing AgBF₄ and stirred for 11 minutes at room temperature, during which time a white precipitate formed. Afterwards, solution was filtered through oven-dried fiberglass filters and the filtrate transferred to an oven-dried 75 mL Hastelloy C autoclave equipped with a stir bar. The stock solution prepared above was then also added to this autoclave (18.489 g, 45.0 mmoles isobutanol), producing a yellow solution, and the autoclave was then sealed and removed from the glovebox. The autoclave was charged with isobutylene (2.3 g, 41 mmoles). The autoclave was then heated to 100° C. and allowed to stir at that temperature at 1000 rpm for 40 hours. The autoclave was then cooled to room temperature, vented and opened. An aliquot was removed from the reactor, filtered through fiberglass and analyzed by GC using the method listed above. GC analysis revealed that the product 2,5-dimethylhex-2-ene had formed, but was present in small amounts. Confirmation of its existence was confirmed by GCMS using selected ion monitoring at 112 and 69 m/z, two of the primary ions formed in its fragmentation. Quantification revealed that the product 2,5-dimethylhex-2-ene formed in <1% selectivity.

### Example 9

### Synthesis of 2,5-dimethylhex-2-ene from isobutanol, isobutylene, (p-cymene)(PCy₃)RuCl₂ and AgBF₄ in the presence of 1,8-Bis(dimethylamino)naphthalene

A stock solution consisting of isobutanol (18.06 wt %), n-decane (1.04 wt %) and chlorobenzene (80.90 wt %) was prepared. The stock solution was sparged with nitrogen 42 minutes. (p-cymene)(PCy₃)RuCl₂ (0.050 g, 0.085 mmoles) was charged into a vial in the glovebox and AgBF₄ (0.033 g, 0.17 mmoles) was added to a separate vial. The ruthenium compound was dissolved in chlorobenzene (2.277 g), producing a red solution. The ruthenium solution was then transferred to the vial containing AgBF₄ and stirred for 11 minutes at room temperature, during which time a white precipitate formed. Afterwards, solution was filtered through oven-dried fiberglass filters and the filtrate transferred to an oven-dried 75 mL Hastelloy C autoclave equipped with a stir bar. The stock solution (18.476 g, 45.0 mmoles isobutanol) prepared above was added to a vial and to this vial was then added 1,8-bis(dimethylamino) naphthalene (0.040 g, 0.19 mmoles). The stock solution containing 1,8-bis(dimethylamino) naphthalene was then transferred to the autoclave and an immediate color change to yellow occurred and a grey precipitate appeared to form. The autoclave was then sealed and removed from the glovebox. The autoclave was then charged with isobutylene (2.5 g, 44 mmoles). The autoclave was then heated to 100° C. and allowed to stir at that temperature at 1000 rpm for 40 hours. The autoclave was then cooled to room temperature, vented and opened. An aliquot was removed from the reactor, filtered through fiberglass and analyzed by GC using the method listed above. GC analysis revealed that the product 2,5-dimethylhex-2-ene had formed, but was present in small amounts. Confirmation of its existence was confirmed by GCMS using selected ion monitoring at 112 and 69 m/z, two of the primary ions formed in its fragmentation. Quantification revealed that the product formed in <1% selectivity.

### Example 10

### Synthesis of 2,5-dimethylhex-2-ene from isobutanol, isobutylene, (p-cymene)(PCy₃)RuCl₂ and AgBF₄ in the presence of 1,8-Bis(dimethylamino)naphthalene at 125° C.

A stock solution consisting of isobutanol (18.06 wt %), n-decane (1.04 wt %) and chlorobenzene (80.90 wt %) was prepared. The stock solution was sparged with nitrogen 42 minutes. (p-cymene)(PCy₃)RuCl₂ (0.050 g, 0.085 mmoles) was charged into a vial in the glovebox and AgBF₄ (0.034 g, 0.17 mmoles) was added to a separate vial. The ruthenium compound was dissolved in chlorobenzene (2.340 g), producing a red solution. The ruthenium solution was then transferred to the vial containing AgBF₄ and stirred for 11 minutes at room temperature, during which time a white precipitate formed. Afterwards, solution was filtered through an oven-dried fiberglass filter and the filtrate transferred to an oven-dried 75 mL Hastelloy C autoclave equipped with a stir bar. The stock solution (19.051 g, 46.4 mmoles isobutanol) prepared above was added to a vial and to this vial was then added 1,8-bis(dimethylamino) naphthalene (0.040 g, 0.19 mmoles). The stock solution containing 1,8-bis(dimethylamino) naphthalene was then transferred to the autoclave and an immediate color change to yellow occurred and a grey precipitate appeared to form. The autoclave was then sealed and removed from the glovebox. The autoclave was then charged with isobutylene (2.1 g, 37 mmoles). The autoclave was then heated to 125° C. and allowed to stir at that temperature at 1000 rpm for 40 hours. The autoclave was then cooled to room temperature, vented and opened. An aliquot was removed from the reactor, filtered through fiberglass and analyzed by GC using the method listed above. GC analysis revealed that the product 2,5-dimethylhex-2-ene had formed, but was present in small amounts. Confirmation of its existence was confirmed by GCMS using selected ion monitoring at 112 and 69 m/z, two of the primary ions formed in its fragmentation. Quantification revealed that the product formed in <1% selectivity.

### Example 11

### Attempted synthesis of 2-ethyl-1H-indene at 90° C.

Indene was filtered through a plug of silica and a stock solution was prepared from the filtered indene. The stock solution consisted of chlorobenzene (92.32 wt %), indene (4.95 wt %) and ethanol (2.73 wt %). The stock solution was degassed by sparging with nitrogen. The ruthenium compound [(C₆H₆)(PCy₃)Ru(H)(CO)][BF₄] (13.0 mg, 0.02 mmoles, from Example 5) prepared above was massed into a vial in the glovebox. To this vial was added 2.55 g of the stock solution (1.5 mmoles of EtOH and 1.1 mmoles of indene). The reaction mixture was then transferred to a 50 mL Schlenk flask equipped with a stir bar. The flask was removed from the glovebox and heated to 90° C. in an oil bath for 5 hours. Afterwards, the solution was filtered through fiberglass and analyzed by GC and GC-MS. The primary product from indene determined in this reaction was indan. The primary ethanol products are acetaldehyde, ethylacetate, diethyl ether and 1,1-diethoxyethane and were identified by GC/MS. 2-ethyl-1H-indene was not observed in the GC chromatogram.

### Example 12

### Synthesis of 2-ethyl-1H-indene at 90° C.

A stock solution consisting of chlorobenzene (93.04 wt %), indene (4.86 wt %) and ethanol (2.09 wt %) was prepared and degassed by 3 freeze/pump/thaw cycles. The stock solution (2.57 g, 1.1 mmoles indene and 1.2 mmoles ethanol) was added to a vial containing the ruthenium compound [(C₆H₆)(PCy₃)Ru(H)(CO)][BF₄] (50 mg, 0.08 mmoles, from Example 4) in the glovebox. The ruthenium compound [(C₆H₆)(PCy₃)Ru(H)(CO)][BF₄] used in this reaction was from a different batch and had a different concentration of the [H—PCy₃][BF₄] impurity, which was present in a 0.6:1.0 molar ratio of [H—PCy₃][BF₄]: [(C₆H₆)(PCy₃)Ru(H)(CO)][BF₄]. The reaction solution was then transferred to a 75 mL Hastelloy C autoclave, which was then assembled and removed from the glovebox. The autoclave was then placed in a 90° C. oil bath and stirred at this temperature for 5 hours. Afterwards, the reaction mixture was filtered through a plug of silica and then analyzed by GC and GC-MS. The compound 2-ethyl-1H-indene was present in the reaction mixture. The mixture was quantified by spiking the sample with a known amount of decane and using the effective carbon numbers to determine the amounts of product formed. In this reaction, the product 2-ethyl-1H-indene formed in <2% selectivity based on converted indene. The primary indene product is indan with >98% selectivity based on indene conversion. The ethanol products formed are primarily acetaldehyde, ethylacetate, diethyl ether and 1,1-diethoxyethane and were identified by GC-MS.

### SPECIFIC EMBODIMENTS

While the following is described in conjunction with specific embodiments, it will be understood that this description is intended to illustrate and not limit the scope of the preceding description and the appended claims.

A first embodiment of the invention is a method of making 2,5-dimethylhex-2-ene comprising reacting isobutene with isobutanol in the presence of a platinum group catalyst to form 2,5-dimethylhex-2-ene. An embodiment of the invention is one, any or all of prior embodiments in this paragraph up through the first embodiment in this paragraph wherein the platinum group metal catalyst comprises a ruthenium catalyst. An embodiment of the invention is one, any or all of prior embodiments in this paragraph up through the first embodiment in this paragraph wherein the ruthenium catalyst comprises Ru/C, Ru/Al₂O₃, or combinations thereof. An embodiment of the invention is one, any or all of prior embodiments in this paragraph up through the first embodiment in this paragraph wherein the ruthenium catalyst comprises [(C₆H₆)(PCy₃)(CO)RuH]⁺BE₄⁻, (PCy₃)₂(CO)RuH(μ-OH)(μ-H)(PCy₃)(CO)RuH and HBF₄Et₂O, or (p-cymene)(PCy₃)RuCl₂ and AgBF₄. An embodiment of the invention is one, any or all of prior embodiments in this paragraph up through the first embodiment in this paragraph wherein the reaction takes place in the presence of a solvent. An embodiment of the invention is one, any or all of prior embodiments in this paragraph up through the first embodiment in this paragraph wherein the solvent is chlorobenzene, isobutanol, dichloromethane or combinations thereof. An embodiment of the invention is one, any or all of prior embodiments in this paragraph up through the first embodiment in this paragraph wherein the reaction has a selectivity of greater than about 25%. An embodiment of the invention is one, any or all of prior embodiments in this paragraph up through the first embodiment in this paragraph wherein the isobutene is derived from a renewable source. An embodiment of the invention is one, any or all of prior embodiments in this paragraph up through the first embodiment in this paragraph wherein the isobutanol is derived from a renewable source.

A second embodiment of the invention is a method of making p-xylene comprising reacting isobutene with isobutanol in the presence of a platinum group metal catalyst to form 2,5-dimethylhex-2-ene; and reforming the 2,5-dimethylhex-2-ene in a reforming zone under reforming conditions to form p-xylene. An embodiment of the invention is one, any or all of prior embodiments in this paragraph up through the second embodiment in this paragraph wherein the platinum group metal catalyst comprises a ruthenium catalyst. An embodiment of the invention is one, any or all of prior embodiments in this paragraph up through the second embodiment in this paragraph wherein the ruthenium catalyst comprises Ru/C, Ru/Al₂O₃, or combinations thereof. An embodiment of the invention is one, any or all of prior embodiments in this paragraph up through the second embodiment in this paragraph wherein the ruthenium catalyst comprises [(C₆H₆)(PCy₃)(CO)RuH]⁺BE₄⁻, (PCy₃)₂(CO)RuH(μ-OH)(μ-H)(PCy₃)(CO)RuH and HBF₄Et₂O, or (p-cymene)(PCy₃)RuCl₂ and AgBF₄. An embodiment of the invention is one, any or all of prior embodiments in this paragraph up through the second embodiment in this paragraph wherein the reaction takes place in the presence of a solvent. An embodiment of the invention is one, any or all of prior embodiments in this paragraph up through the second embodiment in this paragraph wherein the solvent is chlorobenzene, dichloromethane, isobutanol, or combinations thereof. An embodiment of the invention is one, any or all of prior embodiments in this paragraph up through the second embodiment in this paragraph wherein the reaction has a selectivity of greater than about 25%. An embodiment of the invention is one, any or all of prior embodiments in this paragraph up through the second embodiment in this paragraph wherein the isobutene is derived from a renewable source. An embodiment of the invention is one, any or all of prior embodiments in this paragraph up through the second embodiment in this paragraph further comprising dehydrating isobutanol derived from a renewable source to produce the isobutene. An embodiment of the invention is one, any or all of prior embodiments in this paragraph up through the second embodiment in this paragraph wherein the isobutanol is derived from a renewable source. An embodiment of the invention is one, any or all of prior embodiments in this paragraph up through the second embodiment in this paragraph wherein the isobutene and the isobutanol are derived from renewable sources.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

From the foregoing description, one skilled in the art can easily ascertain the essential characteristics of this invention and, without departing from the spirit and scope thereof, can make various changes and modifications of the invention to adapt it to various usages and conditions.

1. A method of making one or more 2,5-dimethylhexenes comprising reacting isobutene with isobutanol in the presence of a platinum group catalyst to form one or more 2,5-dimethylhexenes

2. The method of claim 1 wherein the platinum group metal catalyst comprises a ruthenium catalyst.

3. The method of claim 1 where the reaction occurs at a temperature from about 75 to about 150° C.

4. The method of claim 2 wherein the ruthenium catalyst comprises Ru/C, Ru/Al₂O₃, or a catalyst precursor selected from the group consisting of [(C₆H₆)(PCy₃)(CO)RuH]⁺BE₄⁻, (PCy₃)₂(CO)RuH(μ-OH)(μ-H)(PCy₃)(CO)RuH, {[(PCy₃)(CO)RuH]₄(μ₄-O)(μ₃-OH)(μ₂-OH)} and (p-cymene)(PCy₃)RuCl₂, or combinations thereof and wherein PCy₃ is tricyclohexylphosphine.

5. The method of claim 1 wherein the reaction takes place in the presence of a solvent.

6. The method of claim 5 wherein the solvent is selected from the group consisting of chlorinated solvents, isobutanol and combinations thereof.

7. The method of claim 1 wherein the reaction has a selectivity to one or more 2,5-dimethylhexenes of greater than about 25%.

8. The method of claim 1 wherein the isobutene is derived from a renewable source.

9. The method of claim 1 wherein the isobutanol is derived from a renewable source.

10. A method of making p-xylene comprising:
reacting isobutene with isobutanol in the presence of a platinum group metal catalyst to form one or more 2,5-dimethylhexenes; and
reforming the one or more 2,5-dimethylhexenes in a reforming zone under reforming conditions to form p-xylene.

11. The method of claim 10 wherein the platinum group metal catalyst comprises a ruthenium catalyst.

12. The method of claim 11 wherein the ruthenium catalyst comprises Ru/C, Ru/Al₂O₃, or a catalyst precursor selected from the group consisting of [(C₆H₆)(PCy₃)(CO)RuH]⁺BF₄⁻, (PCy₃)₂(CO)RuH(μ-OH)(μ-H)(PCy₃)(CO)RuH, {[(PCy₃)(CO)RuH]₄(μ₄-O)(μ₃-OH)(μ₂-OH)} and (p-cymene)(PCy₃)RuCl₂, or combinations thereof.

13. The method of claim 10 wherein the reaction takes place in the presence of a solvent.

14. The method of claim 13 wherein the solvent is selected from the group consisting of chlorinated solvents, isobutanol or combinations thereof.

15. The method of claim 10 wherein the reaction has a selectivity to one or more 2,5-dimethylhexenes of greater than about 25%.

16. The method of claim 10 further comprising dehydrating isobutanol derived from a renewable source to produce the isobutene.

17. The method of claim 10 wherein the isobutene or the isobutanol are derived from renewable sources.

18. The method of claim 10 wherein the reforming zone operates at a temperature from about 260° C. to about 600° C. and operates at a pressure from about 100 kPa to about 1.0 MPa.

19. The method of claim 10 wherein the reforming zone operates at a liquid hourly space velocity from about 0.5 hr⁻¹ to about 40 hr⁻¹.

20. The method of claim 10 wherein the hydrogen:hydrocarbon feed molar ratio ranges from about 0.1 to about 10.
